(19) United States
(12) Patent Application Publication
(10) Pub. No.: US 2026/0166321 A1
(43) Pub. Date: Jun. 18, 2026

(54) APPARATUSES AND METHODS FOR SETTING AN ELECTRICAL DOSE

(71) Applicant: Neuros Medical, Inc., Aliso Viejo, CA (US)

(72) Inventors: William R. PATTERSON, Huntington Beach, CA (US); Zi-Ping FANG, Beachwood, OH (US); Nemath SYED SHAH, Lyndhurst, OH (US)

(21) Appl. No.: 19/122,706

(22) PCT Filed: Oct. 19, 2023
(86) PCT No.: PCT/US2023/077351

§ 371 Date: Apr. 18, 2025

Related U.S. Application Data
(60) Provisional application No. 63/417,646, filed on Oct. 19, 2022.

(51) Int. Cl. A61N 1/36

(52) U.S. Cl. CPC A61N 1/36071; A61N 1/36139; A61N 1/3614; A61N 1/36171

(57) ABSTRACT

Methods and apparatuses (e.g., devices, systems, etc.) for applying a therapeutic dose of electrical energy from a nerve cuff on the nerve to modulate nerve function, including reducing pain, may include using a normalized treatment dose value (or range of values) indicating a target therapeutic charge dose to be applied. These apparatuses and methods may use the impedance value of the nerve at the nerve cuff as well as the cross-sectional area of the region of the nerve at least partially surrounded by the nerve cuff in conjunction with the target normalized treatment dose value(s) to determine the parameters for applied energy. In particular, the parameters may be those needed to achieve high-frequency nerve block at the target normalized treatment dose value(s).

### CLAIM OF PRIORITY

This patent application claims priority to U.S. provisional patent application No. 63/417,646, titled “APPARATUSES AND METHODS FOR SETTING AN ELECTRICAL DOSE,” and filed on Oct. 19, 2022.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Implantable neuromodulators (e.g., implantable neurostimulators, implantable nerve block apparatus) are increasingly used to treat pain and other indications, in many cases by the direct application of electrical energy to one or more nerves, including nerve bundles. Such electrical modulation may be used to excite or inhibit nerves, or both. In addition to treating pain, electrical modulation may be applied to a nerve to treat the unwanted and/or uncoordinated generation of nerve impulses which may otherwise be a disabling factor in some medical conditions. An implantable neuromodulator may be implanted on, around or adjacent to a patient's nerve or nerves for the delivery of electrical energy.

Electrical modulation to treat a patient is generally sensitive to the amount, duration and intensity of the applied energy. For example, one non-limiting type of electrical therapy is applying high-frequency alternating current (HFAC) to nerves that have been shown to block nerve activity, e.g., in the treatment of pain. An appropriate dose (e.g., the amount of electrical energy applied to the patient for effective treatment) may be understood to be the amount that causes the desired effect, such as inhibition of nerve activity to reduce pain. An inappropriate dosing may lead to no effect or possibly to irritation of the nerve. Unfortunately, it is generally quite difficult to determine what an appropriate dose is, particularly when dealing with treatment of pain, which may be somewhat subjective and highly variable between individuals. Typically, determining proper dosing for a patient is a time-intensive, and complicated process in which patient-reported, experiential information is required. As a result, the optimal dosing to treat a patient may be highly variable between patients, and indeed, even over time in the same patient. Thus, it would be beneficial to provide a method and/or apparatus for simplifying and reliably setting patient dosing. Described herein are methods and apparatuses that may address these needs.

### SUMMARY OF THE DISCLOSURE

The inventions described herein relate to the field of implantable neuromodulators, and in particular to the field of high-frequency nerve block. These methods and apparatuses may provide a patient-specific pulse profile that is customized to enhance the changes that the applied therapy will result in a significant therapeutic effect. These methods and apparatuses provide, for the first time, techniques that address and may prevent patients from failing to respond to neuromodulation to block pain, including (but not limited to) phantom limb pain. In general, these apparatuses (e.g., systems, devices, etc., including software, firmware and/or hardware) and methods are configured to determine the pulse profile for a high-frequency (e.g., nerve blocking) signal to be applied to a nerve based on a normalized treatment dose value (either a particular, pre-set value, or a range of values). For example, the pulse profile for the high-frequency signal may be estimated by the method or apparatus using a target normalized treatment dose value, an estimate of the cross-sectional area of the region of the nerve under the nerve cuff, and the impedance of the tissue (e.g., nerve) in contact with the nerve cuff.

In general, the normalized treatment dose value is a charge density value that may be delivered, e.g., equivalent to the charge density delivered by a bi-directional waveform (having an associated pulse width and amplitude, in which charge density is equal to the current (e.g., in Coulombs/second or μC/sec) times pulse width (in seconds/phase), divided by the cross-sectional area of the region of the nerve under the nerve cuff. Thus, the charge per phase may be normalized as a dose (“normalized dose”) delivered to a nerve that blocks the conduction of pain signals to the central nervous system and to the brain. For example, the normalized dose values may be between about 0.1 and 5 μC/phase/cm² (e.g., between about 0.2 and 5 μC/phase/cm², between about 0.2 and 3.5 μC/phase/cm², between about 0.3 and 5 μC/phase/cm², between about 0.4 and 5 μC/phase/cm², between about 0.5 and 5 μC/phase/cm², between about 0.1 and 4.5 μC/phase/cm², between about 0.1 and 4 μC/phase/cm², between about 0.1 and 3.5 μC/phase/cm², between about 0.1 and 3 μC/phase/cm², between about 0.1 and 2.5 μC/phase/cm², between about 0.1 and 2 μC/phase/cm², etc.).

For example, described herein are systems for applying a high-frequency nerve block, the system comprising: a nerve cuff comprising one or more electrodes, wherein the nerve cuff is configured to at least partially surround a region of a nerve; a pulse generator configured to generate a high-frequency signal having a pulse profile; a controller configured to determine the pulse profile based on a normalized treatment dose value, a cross-sectional area of the region of the nerve that is at least partially surrounded by the nerve cuff, and an impedance measured from the one or more electrodes, wherein the controller is configured to drive the pulse generator to deliver the high-frequency signal having the pulse profile from the one or more electrodes.

Any of these apparatuses (e.g., systems, devices, etc.) may include an input for determining or receiving either the cross-sectional area of the region of the nerve under the nerve cuff (e.g., transverse to the long axis of the nerve). The controller may be configured to receive data from the input. The cross-sectional area may be entered once (e.g., upon or shortly after implantation) or multiple times, e.g., after healing has occurred, following implantation, and/or periodically thereafter (e.g., every week, every month, every 3 months, every 6 months, every year, etc.).

Any of these systems may include a memory accessible by the controller, the memory configured to store the normalized treatment dose value and/or the cross-sectional area of the region of the nerve that is at least partially surrounded by the nerve cuff. The pulse generator may be configured to generate the high-frequency signal having a frequency of 1 kHz or greater. The pulse generator may be configured to generate the high-frequency signal having a frequency of between 1 kHz and 100 kHz.

In any of these apparatuses at least part of the apparatus (e.g., the implantable controller, pulse generator, memory, etc.) may be enclosed within a housing to be inserted into the body. For example, the apparatus may include a housing enclosing the controller, memory and pulse generator. Thus, the controller may be configured to be implantable. As described in greater detail herein, in some examples a second controller (external controller) may be included as well, which may communicate with the pulse generator and an internal controller. In some examples, the calculation of the pulse profile may be performed internally, using the controller that is enclosed within the housing. Alternatively, in some examples the controller that is determining the pulse profile may be external (e.g., an external controller) that is in communication, e.g., wireless communication (e.g., Wi-Fi, Bluetooth, etc. using a wireless sub-system) with the pulse generator and/or the internal controller. Thus, in any of these systems the controller may be external and may wirelessly communicates with an implant controller coupled to the pulse generator. In some examples the controller(s) may distribute the functions between an internal controller and an external controller. In some examples the internal controller determines the pulse profile and only transmits the pulse profile and/or impedance measurements, etc. for storage, transmission and/or future analysis to an external controller, memory, etc.

The apparatus may receive and/or determine the cross-sectional area. For example, in any of these apparatuses, the controller may be configured to calculate the cross-sectional area from an indicator of the cross-sectional area received by the controller. The indicator of cross-sectional area may include a measure of one or more of diameter, circumference, etc. The indicator may be provided by a medical professional (e.g., doctor, surgeon, nurse, technician, etc.) based on a measure made during the implantation, e.g., using a vessel loop. Thus, any of these apparatuses (e.g., systems) may include one or more inputs configured to receive an indicator of the cross-sectional area of the region of the nerve that is at least partially surrounded by the nerve cuff.

Alternatively or additionally, the indicator of the cross-sectional area may be automatically determined by the apparatus, e.g., based on the status of the nerve cuff. For example, the nerve cuff may include a sensor to detect the indicator directly (e.g., measuring the distance between opposite sides of the nerve cuff and therefore approximating the diameter, estimating or measuring the constricted size of the channel of the nerve cuff when applied to the nerve, and therefore the circumference, etc.). The apparatus may include a module (which may be part of the internal and/or an external controller) for determining the cross-sectional area of the region of the nerve at least partially enclosed by the nerve cuff from the indicator. The indicator and/or the cross-sectional area of the region of the nerve may be stored in a memory that is accessible by the controller or may be part of the controller. The memory may be to store the value of the indicator and/or the cross-sectional area of the region of the nerve in a non-volatile manner, so that even if power is lost the value is retained. The apparatus may backup the value of the cross-sectional area of the region of the nerve and/or the indicator, including backing it up to a remote (e.g., cloud) site.

The same, or a different memory that is accessible by the controller may store the normalized treatment dose value. As mentioned above, the normalized treatment dose value may be a range of values (e.g., between about 0.1 and 5 μC/phase/cm²) or a specific value from within this range e.g., 2 μC/phase/cm².

These apparatuses and methods may be configured so that the patient may trigger the delivery of the high-frequency signal and/or set the duration, and/or stop (e.g., immediate stop) delivery of the high-frequency signal. In some examples the apparatus and method may be configured so that the user may select between one or more different dose values (e.g., select between a number of different discrete or continuously changing normalized treatment dose values). In some examples the apparatus may include an external controller configured to signal the controller to deliver the high-frequency signal. The external controller may include one or more controls (buttons, dials, touchscreen inputs, etc.) that may select on/off (e.g., deliver dose, turn off dose), and optimally may select an input (e.g., “high”, “medium”, “low”, etc.) that may be associated, by the controller, with a normalized treatment dose. Optionally, the user may select the dose duration (e.g., 10 min, 15 min, 20 min, 25 min, 30 min, etc.) and/or may schedule the delivery of the dose (based on a calendar and/or time of day). The external controller may be a dedicated device that wirelessly communicates with the implant, and/or it may include software and/or firmware that may run on a generic device (e.g., a phone, tablet, etc.).

As mentioned, in general, the controller (the internal and/or external controller) may calculate the pulse profile. The pulse profile may refer to one or more characteristics of the high-frequency waveform that is emitted by the pulse generator for delivery by the electrodes of the nerve cuff. The pulse profile may include one or more of: pulse width, pulse amplitude, pulse frequency (within the high-frequency domain specified), and pulse burst duration (e.g., treatment time). Any of these parameters may be fixed or set by the controller, while one or more of the others may be varied/calculated. For example, in some examples the pulse amplitude may be fixed. In some examples the pulse amplitude may be varied. In some examples the controller(s) may calculate the pulse width.

In some examples the apparatus described herein comprises an implant sub-system including the housing enclosing the pulse generator, controller, battery, etc., and a nerve cuff. The nerve cuff may be coupled directly to the housing (or more specifically to the electronics within the housing) or the nerve cuff may be coupled to the housing/electronics by an elongate flexible lead, e.g., a lead coupling the pulse generator to the nerve cuff.

Any of these apparatuses may include an impedance sensing sub-system that is configured to determine the impedance measured from the one or more electrodes. For example, the apparatus may include as part of the internal (e.g., implantable) controller, an impedance-sensing circuit configured to detect the impedance at one or more electrodes of the nerve cuff. The detected impedance (Z) may approximate the real and/or complex impedance.

For example, described herein are systems for applying a high-frequency nerve block that include: an implantable nerve cuff comprising one or more electrodes, wherein the nerve cuff is configured to at least partially surround a region of a nerve; a pulse generator configured to generate a high-frequency signal having a pulse profile; a memory storing a normalized treatment dose value and an indicator of cross-sectional area of the region of the nerve that is at least partially surrounded by the nerve cuff; a controller configured to determine the pulse profile based on the normalized treatment dose value, a cross-sectional area of the region of the nerve that is at least partially surrounded by the nerve cuff, and an impedance measured from the one or more electrodes, wherein the controller is configured to drive the pulse generator to deliver the high-frequency signal having the pulse profile from the one or more electrodes.

Also described herein are methods of setting and/or adjusting a dose of electrical energy to treat a patient using a normalized treatment dose. These methods may be used to set the initial pulse profile (e.g., the parameters for the applied pulsed energy, including in particular the high-frequency pulsed energy used for nerve block). These methods and apparatuses may also be used to adjust the pulse profile. For example, described herein are methods of applying a high-frequency nerve block to treat pain, the method comprising: determining an indicator of a cross-sectional area of a region of a nerve at least partially surrounded by a nerve cuff having one or more electrodes; estimating an impedance value from one or more of the electrodes; determining, in a controller, a pulse profile based on a normalized treatment dose value, the cross-sectional area of the region of the nerve at least partially surrounded by the nerve cuff, and the impedance value; and applying a high-frequency signal having the pulse profile to the nerve.

Any of these methods may include applying the nerve cuff at least partially around the nerve (or in some cases applying the nerve cuff so that it encircles the nerve). The nerve cuff may be implanted with a lead or leadless configuration, along with the pulse generator, internal (e.g., implantable) controller, power source, etc.

In any of these methods the high-frequency signal may have a frequency of about 1 kHz or greater. For example, the high-frequency signal may have a frequency of between 1 kHz and 100 kHz. The frequency may be set (e.g., predetermined) or adjustable.

As mentioned above, the normalized treatment dose value may be selected or predetermined from a range of values. For example, the normalized treatment dose value may be between about 0.1 and 5 μC/phase/cm². In some examples the normalized treatment dose value is between about 0.5 and 4 μC/phase/cm². The minimum valve of the normalized treatment dose value may be about 0.1 μC/phase/cm² or more, about 0.2 μC/phase/cm² or more, about 0.3 μC/phase/cm² or more, about 0.4 μC/phase/cm² or more, about 0.5 μC/phase/cm² or more, about 0.6 μC/phase/cm² or more, etc. The maximum value of the normalized treatment dose value may be about 1.4 μC/phase/cm² or less, about 1.5 μC/phase/cm² or less, about 1.6 μC/phase/cm² or less, about 1.7 μC/phase/cm² or less, about 1.8 μC/phase/cm² or less, about 1.9 μC/phase/cm² or less, about 2.0 μC/phase/cm² or less, about 2.1 μC/phase/cm² or less about 2.2 μC/phase/cm² or less, about 2.3 μC/phase/cm² or less about 2.5 μC/phase/cm² or less, about 3 μC/phase/cm² or less, about 3.5 μC/phase/cm² or less, about 4 μC/phase/cm² or less, about 4.5 μC/phase/cm² or less, about 5 μC/phase/cm² or less, about 5.1 μC/phase/cm² or less, about 5.5 μC/phase/cm² or less, etc.

Any of these methods may include estimating the cross-sectional area of the region of a nerve at least partially surrounded by the nerve cuff from the indicator of the cross-sectional area of the region of the nerve enclosed or partially enclosed by the nerve cuff. In some examples the method may include taking (e.g., measuring) an indicator of the cross-sectional area of the region of the nerve enclosed or partially enclosed by the nerve cuff (e.g., nerve diameter, nerve circumference, etc.). Thus, in any of these methods, determining the indicator of the cross-sectional area of the region of a nerve may comprise measuring a circumference or a diameter of the region of the nerve that is or will be at least partially surrounded by the nerve cuff. The indicator may be directly measured, and/or indirectly determined. Any of these methods may include storing either or both the cross-sectional area of the region of the nerve enclosed or partially enclosed by the nerve cuff or the indicator of the cross-sectional area of the region of the nerve enclosed or partially enclosed by the nerve cuff. This value may be stored in a memory accessible by the controller. The value of the cross-sectional area of the region of the nerve enclosed or partially enclosed by the nerve cuff and/or the indicator of the cross-sectional area of the region of the nerve enclosed or partially enclosed by the nerve cuff may be set one time, upon surgical implantation of the nerve cuff or it may be updated periodically (e.g., particularly when automatically determining it, as described above).

Any of these methods may include receiving a signal from a user to apply the high-frequency signal having the pulse profile to the nerve, and/or to select between a variety of possible applied signals (e.g., “high”, “medium”, “low”, etc.) within the normalized treatment dose value range. The user may control the turning on/off of the applied signal (on demand) and/or may schedule for application of the therapy (e.g., one or more times per day).

Any of these methods may include determining the pulse profile by determining one or more of: pulse width, pulse amplitude, pulse frequency, and pulse burst duration. Applying the high-frequency signal may comprises applying the high-frequency signal for a treatment duration. Any appropriate treatment duration may be used (e.g., treatment duration of 10 minutes or more, 15 minutes or more, 20 minutes or more, 25 minutes or more, 30 minutes or more, 35 minutes or more, etc.). The normalized treatment dose value may be estimated based on a standard treatment duration (e.g., 30 minutes) and/or may be adjusted based on the treatment duration.

For example, a method of applying a high-frequency nerve block to treat pain, the method comprising: applying a nerve cuff comprising one or more electrodes at least partially around a patient's nerve; determining an indicator of a cross-sectional area of a region of the patient's nerve at least partially surrounded by the nerve cuff; estimating an impedance value from one or more of the electrodes; determining, in a controller, a pulse profile based on a normalized treatment dose value of between 0.1 and 5 μC/phase/cm², a cross-sectional area of the region of the patient's nerve at least partially surrounded by the nerve cuff, and the impedance value; and applying a high-frequency signal having the pulse profile to the patient's nerve.

A method (including but not limited to a method of applying a high-frequency nerve block to treat pain) may include: determining or receiving, in a controller for a neurostimulator that is coupled to a nerve cuff comprising one or more electrodes at least partially around a patient's nerve, a cross-sectional area of a region of the patient's nerve at least partially surrounded by the nerve cuff; determining an impedance value from one or more of the electrodes; determining, in a controller, a pulse profile based on a normalized treatment dose value of between 0.1 and 5 μC/phase/cm², the cross-sectional area of the region of the patient's nerve at least partially surrounded by the nerve cuff, and the impedance value; and applying a high-frequency signal having the pulse profile to the patient's nerve.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 shows a graph illustrating the percentage of patient sessions with at least 30% pain reduction immediately after 30 min of treatment with a single nerve cuff as a function of a normalized dose (in μC/phase/cm²) for all subject of a clinical trial (QUEST RCT Treatment subjects, n=85). The “absolute” non-responders (e.g., 0% of sessions when dose ranges from >=2) were included, although it is likely that these patients may represent false positives during lidocaine screening.

FIG. 2 is a graph illustrating the percent of patient sessions with at least 50% pain reduction at 120 min post-treatment with a single nerve cuff as a function of a normalized dose (in μC/phase/cm²) for treatment subjects (n=85).

FIG. 3A shows one example of a neuromodulation system (showing a nerve cuff, lead and implantable controller/waveform generator that may include the dose-setting controller).

FIG. 3B shows an example of the system of FIG. 3A implanted into a patient, also showing a dose-setting controller, in this example, an optional external controller, which may communicate with the implant. The optional external controller (also referred to herein as a patient controller or user controller) may include or communicate with a dose selector driving the application of energy by the implant.

FIG. 3C is a schematic illustration of a system for treating a patient's pain using high-frequency nerve block that includes a dose-setting controller as described herein.

FIG. 4 schematically illustrates one example of a system for treating a patient's pain including normalized dose setting.

FIG. 5 is a flow diagram illustrating one example of a method of treating a patient's pain by delivering neuromodulation from an implant using a normalized dose as described herein.

### DETAILED DESCRIPTION

Methods and apparatuses (e.g., devices, systems, etc.) for applying a therapeutic dose of electrical energy from a nerve cuff on the nerve to modulate nerve function, including reducing pain, may include using a normalized treatment dose value (or range of values) indicating a target therapeutic charge dose to be applied. These apparatuses and methods may use the impedance value of the nerve at the nerve cuff as well as the cross-sectional area of the region of the nerve at least partially surrounded by the nerve cuff in conjunction with the target normalized treatment dose value(s) to determine the parameters for applied energy. In particular, the parameters may be those needed to achieve high-frequency nerve block at the target normalized treatment dose value(s).

For example, any of the methods and apparatuses described herein may include implanting a patient with a nerve cuff electrode in direct electrical contact with a nerve. The nerve cuff electrode may partially or completely encircle the nerve. The nerve cuff electrode may be connected to a pulse generator (PG) configured to produce a bi-directional waveform with amplitude controlled by voltage or by current. The pulse generator may be configured to transmit and receive data to and from a processor (e.g., computer) through a communication software protocols. In any of these apparatuses the pulse generator may be configured to receive the impedance (Z) measured at the nerve cuff electrode/nerve interface, and the pulse generator may be configured to transmit increases or decreases in voltage or in current to the nerve cuff electrode/nerve interface. Any of these apparatuses may be configured to determine and/or store the nerve cross-sectional area for the region of the nerve that is encircled (or at least partially encircled) by the cuff. In general, these apparatuses may be configured to respond to a measured impedance (Z), a change in voltage or in current changes the charge density delivered by the bi-directional waveform (with its associated pulse width) according to the following equation:

$$Charge Density = \frac{Current ( \frac{Coulomb}{second} ) \times Pulse Width ( \frac{second}{phase} )}{Nerve Cross - sectional Area ( {cm}^{2} )}$$

The charge per phase may be normalized as a dose (“normalized dose”) delivered to a nerve that blocks the conduction of pain signals to the central nervous system and to the brain. As shown in equation (1), the normalized dose may be in units of C/(phase*cm²), or more likely microC/(phase*cm²), e.g., μC/phase/cm².

As described in detail herein, by normalizing the dose to the cross-sectional area of the region of the nerve surrounded by the nerve cuff, the response may be both more predictable and graded, in a manner similar to a pharmaceutical dose. This may provide a number of benefits to both patients and caregivers, including the ability to more precisely treat a variety of different patients, and prevent acclimation, reduce side-effects and prevent or limit desensitization.

Surprisingly, the methods and apparatuses described herein may also provide an electrical treatment of pain in a manner that is analogous to pain relief from pharmaceutical pain medications, in which increasing doses (e.g., below intolerable side-effect thresholds) also increases the level of pain relief experienced by the patient. In general, the methods and apparatuses described herein provide treatments in which the charge density may be set for initial dosing and may be increased or decreased in a controlled manner to modulate pain reduction. Pain reduction may be measured by patient-reported outcomes (PRO).

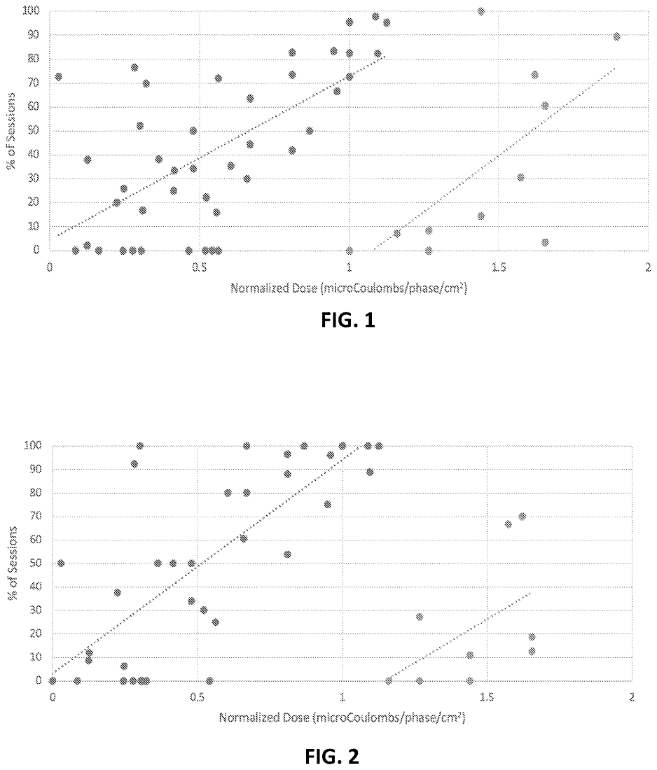

For example, in FIG. 1 illustrates the results of a recent trial using a nerve-cuff apparatus similar to that described herein. In this trial (“QUEST”), lower-limb amputee patients with chronic and severe residual limb pain or phantom limb pain who attained significant pain reduction after local nerve block injection (e.g., lidocaine injection) were enrolled. A single cuff electrode was wrapped around the sciatic nerve or two cuffs (1 each) on the tibial and common peroneal nerves, e.g., the above the knee or below the knee. An implantable generator was used to apply sinusoidal waveforms of 5 kHz or 10 kHz and up to 16 V or 20 mA were applied for 30 min intervals during each subject-initiated treatment session. A diary was used to record pain intensities before and after each session.

The dosing in the QUEST trial was initially calibrated as suggested in the art. See, e.g., PCT/US2019/047281 (filed Aug. 20, 2019), titled “APPARATUSES AND METHODS FOR ADJUSTING A THERAPEUTIC ELECTRICAL DOSE,” and corresponding U.S. patent application Ser. No. 17/265,532. Initial calibration used patient feedback during an initial calibration period, requiring a technician to set the initial dosing levels as well as patient participation. Selected and applied voltages and impedances were determined, in additional to other patient specific data, including anatomic, age and other related parameters. The results of the QUEST trial were analyzed, as described herein. Only by analyzing the results of multiple patient groups (including both responders and non-responders) at different apparent effectiveness levels (e.g., various percentage of pain reduction at differing time points), and only after adjusting applied energy to a normalized dose that accounts for the area of the nerve that is at least partially encircled by the nerve cuff, was it apparent that the effectiveness of the pain reduction across different patients could be titrated based on a normalized dose, as described herein.

For example, eighty-five (85) subjects from the QUEST trial were analyzed based on the percent of their PRO sessions that achieved at least 30% pain reduction after a 30 min delivery of electricity as a function of their charge density or “dose.” FIG. 1 shows a positive linear dependence or “dose response effect” in 2 separate groups of subjects with a larger electrical dose (between ˜0.75 and 1 and between ˜1.5 and 2). Two example (dashed) lines are shown approximating a linear regression of responding patients. Subjects in the above ranges had 30% pain reduction in 70% or more of their sessions with immediately after a 30 min session.

The pharmaceutical “dose response effect” of electricity delivered to a nerve is further corroborated by the data shown in FIG. 2. In this example, the patient-reported outcomes data are presented as the percent of sessions with at least 50% pain reduction at 120 min after the original 30 min sessions in FIG. 1. That is, these subjects received one 30 min electrical dose and then record their pain immediately after the session (FIG. 1) and then 120 min later (FIG. 2), showing the persistence of the effect.

In general, the electrical dose, in a single session (e.g., of 30 min) may provide an immediate and moderately important pain reduction of at least 30% in the dose ranges noted above (FIG. 1). In those same dose ranges at 120 min (FIG. 2), the pain reduction improvement is now substantial (at least 50%). Moderately important and substantial improvements in pain reduction are highly clinically significant. A reduction in pain intensity of 10-20% is considered to be a “minimally important” pain intensity reduction, a ≥30% reduction corresponds to what patients would consider a “moderately important” improvement in pain intensity, whereas reductions of approximately 50% or more can be considered “substantial” improvements in pain intensity for individuals with acute and chronic pain. Percentage reduction in pain is generally considered a useful approach to determining whether a patient has had a meaningful improvement. Preliminary results also suggest that these dose-dependent decreases in pain intensity, do not result in significant adverse effects, such as disruption of sleep, mood, and function and therefore offer a major benefit to the patient.

The resulting data and the invention of an electrical dose for pain conduction block can be used to create a baseline for a given patient's starting dose. As mentioned above, it is currently required that the patient, typically shortly after or during the implantation process, is tested by a trained technician during a necessary calibration process in order to set and adjust the treatment baseline. For example, a patient must be moved from 0 volts to progressively increased voltages in an interrogative discussion with a knowledgeable programmer who slowly increases the voltage to a threshold that the patient can tolerate for 30 min. This time- and resource-invasive method is necessary to set the initial therapeutic intensity and may be used to program treatments.

In contrast the methods and apparatuses described herein may instead control and set the therapeutic dose using a measure of the cross-sectional diameter of the region of the nerve (e.g., sciatic, tibial, etc.) nerve enclosed by the nerve cuff as well as the impedance of the nerve/cuff tissue interface region to determine the therapeutic dose, and may set the therapeutic dose within the target range (e.g., between about 0.1 and 5 μC/phase/cm², between 0.2 and 5 μC/phase/cm², 0.2 and 3.5 μC/phase/cm², between about 0.3 and 5 μC/phase/cm², between about 0.4 and 5 μC/phase/cm² etc, between about 0.5 and 5 μC/phase/cm², between about 0.1 and 4.5 μC/phase/cm², between about 0.1 and 4 μC/phase/cm², between about 0.1 and 3.5 μC/phase/cm², between about 0.1 and 3 μC/phase/cm², between about 0.1 and 2.5 μC/phase/cm², between about 0.1 and 2 μC/phase/cm², etc.). Thus, for a given patent having a cross-sectional area of the portion of the nerve enclosed by the nerve cuff, while monitoring the impedance of the nerve cuff (electrode) at the tissue (e.g., at the nerve), the dose may be set to be within the therapeutic dose by setting the pulsing parameters (e.g., pulse width, amplitude, etc.).

With knowledge of the patient's nerve diameter and the nerve cuff/nerve impedance, the range of the normalized dose may be set (e.g., using data such as that shown in FIGS. 1 and 2). This will allow the programmer to optimally position the voltage (and by association the electrical dose) in a narrow range that is known to produce moderately important to substantial improvements in pain relief.

Thus, apparatuses and methods using a therapeutic dose as described herein may provide a range of applied energies that are specific to the particular patient (based on the relationship between the impedance, cross-sectional area of the nerve at the nerve cuff) to provide effective treatment. The initial treatment parameter (e.g., applied energy) may be set to be within the normalized treatment dose value range.

Although the data in FIGS. 1 and 2 are specific for patients for whom a single cuff was applied, similar results are seen in patients for whom two cuffs were used (e.g., applied on both the tibial and common peroneal nerves). Thus, the methods and apparatuses described herein may be used for two (or more) nerve cuffs applied to the same patient. In some examples the combination of the effects of both nerve cuffs, including the cross-sectional areas, and impedances at each of the nerves surrounded (or partially surrounded) by each nerve cuff may be used with the same (or in some examples, a scaled or distributed) normalized treatment dose. For example, the same normalized treatment dose value may be used to determine the pulse profile applied at each of the nerve cuffs, using the impedance and cross-sectional areas of the regions of the nerves at least partially surrounded by a nerve cuffs. Alternatively, the normalized treatment dose may be distributed between the nerve cuffs based on, e.g., a ratio of the cross-sectional areas and/or impedance values of each nerve at the various nerve cuffs.

### Apparatuses

The apparatuses described herein typically include a controller that may determine the parameters (e.g., pulse profile) of the applied energy so that the applied energy is within a predetermined range of normalized treatment dose values (and/or to target a specific normalized treatment dose value), as well as a nerve cuff to at least partially surround the nerve to which the energy is applied. For example, an apparatus may generally include a nerve cuff having one or more electrodes (e.g., an array of electrodes), an implantable pulse generator configured to generate a high-frequency signal having a pulse profile and a controller configured to determine the pulse profile based on a normalized treatment dose value (e.g., the charge delivered). The controller may therefore be configured to determine the pulse profile based on a normalized treatment dose value, a cross-sectional area of the region of the nerve that is at least partially surrounded by the nerve cuff, and an impedance measured from the one or more electrodes, wherein the controller is configured to drive the pulse generator to deliver the high-frequency signal having the pulse profile from the one or more electrodes.

These apparatuses (e.g., systems, devices, and software, including neuromodulators and neuromodulation systems) may be configured for treating a patient's pain and may include a one or more sub-systems or modules that may be hardware, software and/or firmware, for determining the energy applied for treatment (e.g., pulse profile) so that the energy delivered is within a target normalized treatment dose value range (and/or is at the normalized treatment dose value).

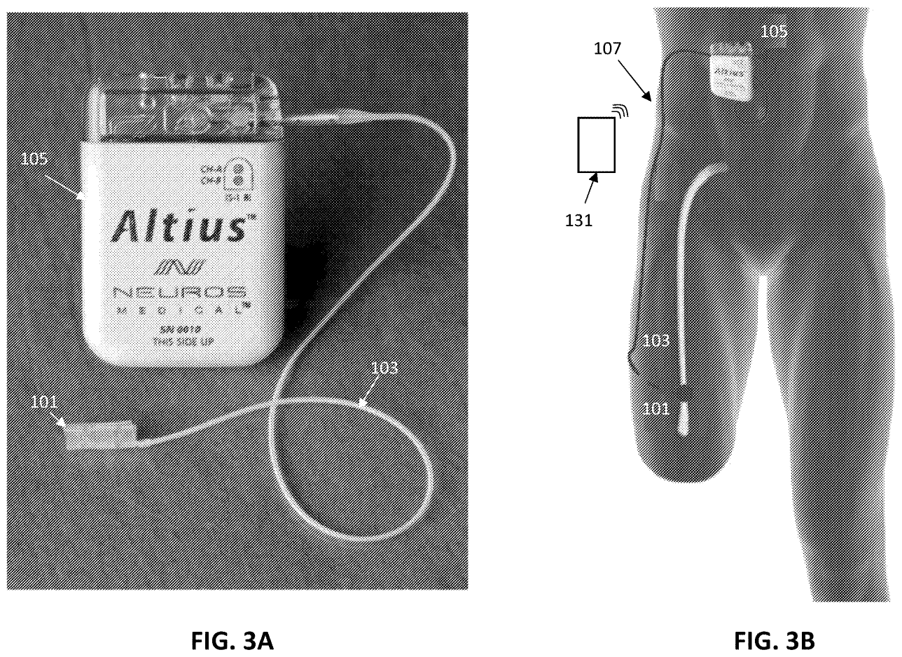

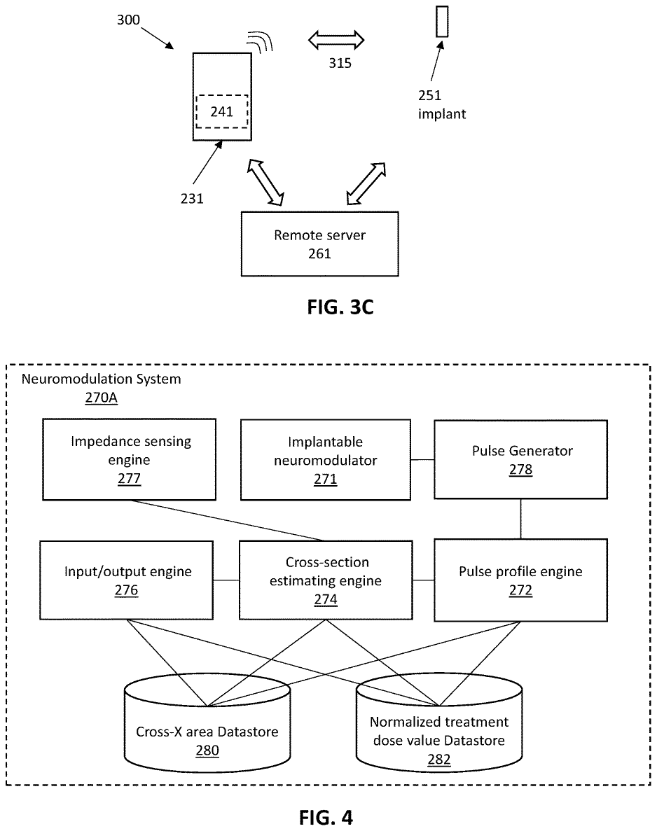

These methods and apparatuses may be used with any appropriate neuromodulator. FIG. 3A illustrates one example of an implantable neuromodulator including a nerve cuff 101, a lead 103 connecting the nerve cuff to a controller (e.g., waveform generator, control circuitry, power source, communications circuitry and/or antenna, etc.) within an implantable housing 105. Systems including a nerve cuff such as those described herein, may be used, for example, to apply a high frequency nerve block to acutely treat pain, either acute pain or chronic pain (more than 6 months in duration), in humans by blocking nerve conduction on an action potential. Acute treatment may refer to on-demand treatment with substantially immediate pain relief effect. The nerve cuff may be applied onto a moderate and relatively large diameter nerves such as, but not limited to, the sciatic nerve. One therapy involves reversibly blocking peripheral nerves by applying high frequency alternating current directly on a nerve trunk. A current ranging from 1 kHz to 100 kHz (e.g., 5 kHz to 50 kHz) may be applied; this may be referred to as a high frequency modulation, compared to a current of less than 1 kHz applied in the conventional electrical modulation. Efficacy of the high frequency alternating current therapy in acute non-human animal experiments (frog, cat) has been reported. U.S. Pat. Nos. 7,389,145 and 8,060,208 describe in general this electrical modulation technology.

The nerve cuffs may encircle a particular segment of a targeted peripheral nerve, e.g., a sciatic nerve, a tibial nerve, etc. Using an implanted electrode connected to an electrical waveform generator, an electrical waveform may be applied for a time interval, e.g., 10 min (15 min, 20 min, 25 min, 30 min, 35 min, 40 min, etc.), sufficient to effect substantially immediate patient pain relief, e.g., within 10 min, and an extended period of pain relief up to several hours. The current may be determined as described herein so that the total energy (charge) delivered approximates the target normalized treatment dose value(s) (including being within a range of normalized treatment dose values).

The application of 10 kHz alternating current generated by a custom generator via a custom implanted nerve electrode may significantly reduce pain in the majority of patients treated. For example, an implantable electrode operatively connected to an external or implanted waveform generator may be used. The electrode may be a spiral cuff electrode similar to that described in U.S. Pat. No. 4,602,624. The electrode may be implanted in a human mammal on a desired peripheral nerve trunk proximal to the pain source (e.g., a neuroma), such that the cuff encircled the desired peripheral nerve in which the action potential was to be blocked. The cuff inner diameter may range from about 4 mm to about 13 mm. The sciatic nerve is known to have a relatively large nerve trunk; the diameter of the proximal part of the sciatic nerve may vary between individuals. In one embodiment, the apparatus and method was used on the sciatic nerve to treat limb pain in above knee amputees. In one embodiment, the apparatus and method was used on the tibial nerve to treat limb pain in below knee amputees.

FIG. 3B illustrates the use of a system including a cuff electrode applied to the sciatic nerve of an amputee patient. In this example, the amputee 107 has been implanted with a nerve cuff 101 around the sciatic nerve (nerve trunk), and is connected, via a lead 103, to the controller including the pulse generator (also referred to herein as a waveform generator) within an implantable housing 105. This procedure may be done, for example, by first dissecting to expose the nerve in an open procedure, then wrapping the nerve with the flexible (self-closing) cuff. During the procedure the diameter and/or circumference of the nerve onto which the cuff is implanted may be measured directly or indirectly. This value may be transmitted to the implanted controller and/or to an external controller. Once implanted the controller/waveform generator may be placed in a pocket in the anterolateral abdominal wall, and a tunneling electrode cable may be positioned along the midaxillary line (including transversely across the abdomen) to connect the controller/waveform generator to the nerve cuff electrode. The impedance of the nerve cuff may be determined (e.g., by the system) and the incisions may be closed. The incision for implanting the nerve cuff may be larger than about 1.5 inches (e.g., between 1.5 and 3 inches), so that sufficient visualization and access may be achieved. Once implanted and allowed to heal, the implanted neuromodulator may be set as described herein to provide a therapeutic dose (e.g., an optimized dose) as described herein.

The system shown in FIG. 3B also includes an external controller 131 (e.g., patient controller) that include one or more processors and may be configured to perform at least a portion of the methods described herein. The controller, or a separate device coupled to the controller, may include an input for the user to control. The controller may be software (such as application software) that runs on a personal device, such as a smartphone, table, etc., and may include one or more user interfaces to allow the user to control the operation of the implant.

FIG. 3C schematically illustrates one example of a system 300 (e.g., a system for applying a high-frequency nerve block). The system includes an implant 251 with a nerve cuff comprising one or more electrodes. The nerve cuff is configured to at least partially surround a region of a nerve. The implant 251 also includes a pulse generator configured to generate a high-frequency signal having a pulse profile and a controller configured to determine the pulse profile based on a normalized treatment dose value range. The implant may be configured to wirelessly communicate with an external controller 231 that may include one or more user (e.g., patient, physician, technician, etc.) controls 241, such as a touchscreen, knob, button, etc. The external controller 231 may also include one or more outputs (e.g., LEDs, displays, speaker, etc.). In some examples the external controller is software running on a smartwatch, phone, tablet, etc. The implant and the external controller may communicate with each other 315 (e.g., via a wireless communication protocol and/or sub-system. Either or both the implant and the external controller may also or alternatively communicate with a remote server 261 that may also receive inputs and provide outputs to the implant and/or external controller.

As mentioned above the implantable controller and/or the external controller may be used to determine the pulse profile from the normalized treatment dose value(s). The pulse profile may correspond to the delivered neuromodulation dose and may include a variety of dose parameters for treating pain. In general, a set of dose parameters may include a dose duration (e.g., time the dose is being delivered, which may be the total duration or may be a portion of the total duration, and treatment time), dose frequency (e.g., treatment frequency; in high-frequency never block variations the frequency may be greater than 1 kHz, such as between 1-100 kHz), and peak voltage (e.g., peak modulation voltage, such as between 0.1 V and 20 V, e.g., between 5 V and 15 V, etc.). In some variation the dose parameters may include a therapy ramp-up time to reach a peak modulation voltage and a sustained peak modulation time during which the voltage is sustained at the peak modulation voltage (the dose duration may include both ramp-up time and peak modulation time). The dose parameters may also include the waveform parameters applied, e.g., pulsatile or repeating (e.g., sinusoidal, square wave, saw-tooth, biphasic, etc.), and the frequency of the applied waveform (e.g., the high-frequency component). Other dose parameters may include the initial (e.g., starting) voltage, which may be, e.g., zero, or may be an offset (e.g., voltage offset) voltage. In some variations, the therapeutic dose parameters may include pulse duration (in treatment variations including bursting/pulses), burst duration (in variations including bursting/pulses), pulse shape (e.g., square, triangular, sinusoidal, etc.), biphasic/monophasic (positive and/or negative), carrier frequency (in variations using a carrier frequency), DC offset level (in variations including a DC offset), current level (in variations modulating current), current limit (in variations limiting current), electrode number/location (in variations having more than one pair of electrodes), etc.

In general, the controller (either or both the internal controller in the implant 251 and/or the external controller 231) may be configured to determine the pulse profile in order to determine the delivered treatment dose so that the treatment dose (charge delivered) is within approximately the normalized treatment dose value (or within the normalized treatment dose value(s) range).

FIG. 4 is a diagram showing an example of a neuromodulation system 270A that is configured to set the therapeutic dose based on a normalized treatment dose value as described above. The modules of the neuromodulation system 270A may include one or more modules (which may be referred to herein as sub-systems or engines) and one or more datastores (e.g., memory). The modules may include hardware, firmware and/or software and may be part of the control (e.g., a processor, memory, circuitry, etc.). A module (e.g., engine/sub-system) can be implemented as part of a controller with one or more processors, or one or more modules can be implemented as part of the same (or multiple) controllers. Thus, as used herein a module (engines/sub-systems) may include one or more processors or a portion thereof. A portion of one or more processors can include some portion of hardware less than all of the hardware comprising any given one or more processors, such as a subset of registers, the portion of the processor dedicated to one or more threads of a multi-threaded processor, a time slice during which the processor is wholly or partially dedicated to carrying out part of the engine's functionality, or the like. As such, a first engine and a second engine can have one or more dedicated processors, or a first engine and a second engine can share one or more processors with one another or other engines. Depending upon implementation-specific or other considerations, an engine can be centralized, or its functionality distributed. Thus, an engine can include hardware, firmware, or software embodied in a computer-readable medium for execution by the processor. The processor transforms data into new data using implemented data structures and methods, such as is described with reference to the figures herein.

The engines described herein, or the engines through which the systems and devices described herein can be implemented, can be local or cloud-based engines. As used herein, a cloud-based engine is an engine that can run applications and/or functionalities using a cloud-based computing system. All or portions of the applications and/or functionalities can be distributed across multiple computing devices and need not be restricted to only one computing device. In some embodiments, the cloud-based engines can execute functionalities and/or modules that end users access through a web browser or container application without having the functionalities and/or modules installed locally on the end-users' computing devices.

As used herein, a memory, which may be equivalently referred to as a datastore, is intended to include one or more repositories having any applicable organization of data, including tables, comma-separated values (CSV) files, traditional databases (e.g., SQL), or other applicable known or convenient organizational formats. Datastores can be implemented, for example, as software embodied in a physical computer-readable medium on a specific-purpose machine, in firmware, in hardware, in a combination thereof, or in an applicable known or convenient device or system. A database may be a datastore or part of a datastore. Datastore-associated components, such as database interfaces, can be considered “part of” a datastore, part of some other system component, or a combination thereof, though the physical location and other characteristics of datastore-associated components is not critical for an understanding of the techniques described herein.

Datastores can include data structures. As used herein, a data structure is associated with a particular way of storing and organizing data in a computer so that it can be used efficiently within a given context. Data structures are generally based on the ability of a computer to fetch and store data at any place in its memory, specified by an address, a bit string that can be itself stored in memory and manipulated by the program. Thus, some data structures are based on computing the addresses of data items with arithmetic operations; while other data structures are based on storing addresses of data items within the structure itself. Many data structures use both principles, sometimes combined in non-trivial ways. The implementation of a data structure usually entails writing a set of procedures that create and manipulate instances of that structure. The datastores, described herein, can be cloud-based datastores. A cloud-based datastore is a datastore that is compatible with cloud-based computing systems and engines.

The neuromodulator system 270A may include a computer-readable medium, an implantable neuromodulator 271, and one or more pulse generators 278, one or more datastores (e.g., memory) for holding a normalized treatment dose value(s), or range/ranges of values, e.g., a normalized treatment dose value datastore 282. The same of a different datastore 280 may be used to hold either or both an indicator of a cross-sectional area of the region of the nerve that is at least partially surrounded by the nerve cuff (e.g., the nerve circumference, nerve diameter, etc.) and/or the cross-sectional area of the region of the nerve that is at least partially surrounded by the nerve cuff. The system may include an input/output engine 276 for entering the indicator of the cross-sectional area of the region of the nerve that is at least partially surrounded by the nerve cuff and/or the cross-sectional area of the region of the nerve that is at least partially surrounded by the nerve cuff. The input/output engine may include or may involve wireless communication to/from the controller. In some examples the system includes a cross-section estimating engine 274 for estimating the cross-sectional area of the region of the nerve that is at least partially surrounded by the nerve cuff from the indicator. In some examples this cross-section estimating engine may be configured to receive input from the nerve cuff indicating the cross-sectional area of the region of the nerve that is at least partially surrounded by the nerve cuff. The system may also include an impedance sensing engine 277 to determine the impedance form of the electrode/tissue interface from the nerve cuff.

The systems described herein may also include a pulse profile generating engine 272 that is configured to determine the pulse profile based on the normalized treatment dose value (range(s)) the cross-sectional area of the region of the nerve that is at least partially surrounded by the nerve cuff (or, equivalently, from the indicator of the cross-sectional area of the region of the nerve that is at least partially surrounded by the nerve cuff) and the impedance (from the impedance sensing engine). The pulse profile engine may therefore output one or more parameters for setting the pulse waveform(s) to the pulse generator 278, to drive the pulse generator 278 to deliver the high-frequency signal having the pulse profile from the one or more electrodes of the nerve cuff of the implantable neuromodulator 271.

The implantable neuromodulator may be implanted in the patient (as shown in FIG. 3B) and may communicate with the other components of the system. The input/output engine 276 may also allow the patient to adjust the dose, including switching to a different normalized treatment dose value ranges, as described above. In some variations the pulse profile engine is integrated into or part of the patient controller or may communicate with the external controller to allow selection of different normalized treatment dose values.

In practice the neuromodulation system may set the initial, e.g., starting, dose for the patient as described above, based on the normalized treatment dose value(s). Thereafter, in some examples, subsequent doses may be based on this initial dose. Alternatively or additionally, subsequent doses may be recalculated, as the impedance may change and/or the target normalized treatment dose value may change. For example the user may increase or decrease the intensity desired, which may be adjusted by selecting more or less charge within the range of the acceptable normalized treatment dose values.

### Methods

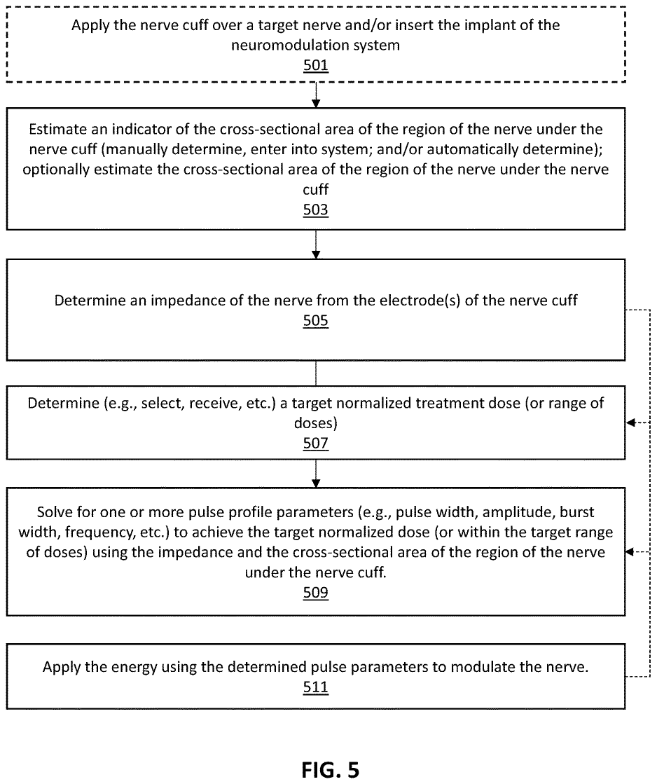

FIG. 5 illustrates an example of a method of treating a patient including setting the treatment dose using the normalized treatment dose value (or range of normalized treatment dose values) to determine the parameters for applying the therapy. Optionally in some examples the nerve cuff may be implanted at least partially over a target nerve (e.g., a sciatic nerve, a tibial nerve, etc.) 501. The nerve cuff may be completely or partially wrapped around a portion of the nerve. An indicator of the cross-sectional area, such as the diameter and/or the circumference of the region of the never enclosed (or partially enclosed) by the nerve cuff may then be estimated, providing a cross-sectional area of the region of the nerve that is at least partially surrounded by the nerve cuff 503. The cross-sectional area of the region of the nerve that is at least partially surrounded by the nerve cuff may be determined automatically and/or manually. For example the clinician may measure or approximate the circumference or diameter of the nerve when applying the nerve cuff and may enter this indicator so that the system may calculate the cross-sectional area of the region of the nerve that is at least partially surrounded by the nerve cuff. Alternatively the clinician my calculate and enter the cross-sectional area of the region of the nerve that is at least partially surrounded by the nerve cuff. In some examples the apparatus may automatically estimate the cross-sectional area of the region of the nerve that is at least partially surrounded by the nerve cuff.

In any of these methods, an impedance of the one or more electrodes on the tissue may be determined from the one or more electrodes on the nerve cuff 505. In some examples the impedance is examined as part of the implantation process for applying the nerve cuff to the nerve to confirm good contact with the nerve. Optionally, the impedance may be detected as part of the implant (e.g., part of the neuromodulation system, such as part of an impedance sensing engine).

The method may include selecting or determining a target normalized treatment dose 507 (or range of doses). For example, the method may include using a predetermined normalized treatment dose or receiving a user-selected normalized treatment dose (e.g., from an external controller). The normalized treatment dose selected or determined may then be used to solve for the parameters for applying energy (e.g., charge) to a nerve having the measured cross-sectional area when the impedance is as determined 509. Some of the parameters (e.g., amplitude, frequency, burst duration, etc.) may be preset, while others (e.g., pulse width) may be solved to determine what their value should be to achieve the target normalized treatment dose within the permitted range of normalized treatment doses. Any of the stimulation parameters may be varied while the remaining parameters are fixed to preset values.

As shown in FIG. 5, once the treatment parameters have been determined from the target normalized treatment dose, impedance and cross-sectional area of the nerve under the nerve cuff, the energy may be applied, via the nerve cuff, to treat the patient 511. Thereafter, e.g., after a sufficient “off” time, and/or as decided by the patient, in an on-demand system, the process may be repeated, optionally from the step of determining the impedance 505, or determining the target normalized treatment dose 507, or by solving for the one or more pulse parameters 509. In some cases the same pulse parameters may be used on a timed or repeating scheduled (or upon demand from the patient).

In any of the methods and apparatuses described herein the pulse parameters (and normalized dose) may refer to volume, rather than cross-sectional area. For example, the volume of the region of the nerve under the electrodes, rather than the cross-sectional area, may be used by the controller to determine the pulse profile. For example, the controller may determine the pulse profile based on the normalized treatment dose value, a volume of the region of the nerve that is at least partially surrounded by the nerve cuff, and an impedance measured from the one or more electrodes. The volume may be determined by multiplying the cross-sectional area of the nerve (as describe above) by the length of the nerve covered by the nerve cuff, or in some variations, the length of the nerve in contact with the electrodes within the nerve cuff. Any of these methods and apparatuses may therefore refer to charge per volume rather than change per cross-sectional area (e.g., cm³ rather than cm²).

It should be understood that the methods and apparatuses described herein typically refer to cross-sectional area of the underlying nerve being targeted by the electrodes and not the cross-sectional area of the electrodes. The cross-sectional area of the electrodes that is used when describing current density (or in some cases charge density), which may refer to the electrochemical properties of the unit surface are of the electrode, is not the same as the cross-sectional length (or volume) of the nerve at least partially surrounded by the nerve cuff described herein. Thus, the use of the cross-sectional area of the nerve is not equivalent to the more traditional use cross-sectional area of the electrodes, traditionally used when estimating electrical charge density of an electrode.

In any of the apparatuses described herein (or methods for using them), the apparatus may include one or more inputs for manually entering the cross-sectional area (or in some examples, volume) of the region of the nerve under the cuff. Alternatively or additionally, the apparatus may automatically estimate or determine the cross-sectional area (or volume) of the region of the nerve under the cuff—e.g., the cross-sectional area of the region of the nerve that is at least partially surrounded by the nerve cuff.

The controller described herein may include a memory (e.g., storing the software/programs described above for determining the pulse parameters to be applied, and/or controlling the application. The controller may include or may be coupled with the pulse generator; in some examples the pulse generator may include the controller and/or the two may be collectively referred to as the pulse generator (PG) or implantable pulse generator (IPG), in variations that are fully implanted.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, any of the methods described herein may be performed, at least in part, by an apparatus including one or more processors having a memory storing a non-transitory computer-readable storage medium storing a set of instructions for the processes(s) of the method.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A system for applying a high-frequency nerve block, the system comprising:
a nerve cuff comprising one or more electrodes, wherein the nerve cuff is configured to at least partially surround a region of a nerve;
a pulse generator configured to generate a high-frequency signal having a pulse profile;
a controller configured to determine the pulse profile based on a normalized treatment dose value, a cross-sectional area of the region of the nerve that is at least partially surrounded by the nerve cuff, and an impedance measured from the one or more electrodes, wherein the controller is configured to drive the pulse generator to deliver the high-frequency signal having the pulse profile from the one or more electrodes.

2. The system of claim 1, further comprising a memory accessible by the controller, the memory configured to store the normalized treatment dose value and/or the cross-sectional area of the region of the nerve that is at least partially surrounded by the nerve cuff.

3. The system of claim 1, wherein the pulse generator is configured to generate the high-frequency signal having a frequency of 1 kHz or greater.

4. The system of claim 1, wherein the pulse generator is configured to generate the high-frequency signal having a frequency of between 1 kHz and 100 kHz.

5. The system of claim 1, further comprising a housing enclosing the controller, memory and pulse generator.

6. The system of claim 1, wherein the controller is implantable.

7. The system of claim 1, wherein the controller is external and wirelessly communicates with an implant controller coupled to the pulse generator.

8. The system of claim 1, wherein the controller is configured to calculate the cross-sectional area from an indicator of the cross-sectional area received by the controller.

9. The system of claim 1, further comprising an input configured to receive an indicator of the cross-sectional area of the region of the nerve that is at least partially surrounded by the nerve cuff.

10. The system of claim 1, wherein the normalized treatment dose value is between 0.1 and 5 μC/phase/cm².

11. The system of claim 1, further comprising an external controller configured to signal the controller to deliver the high-frequency signal.

12. The system of claim 1, wherein the pulse profile comprises one or more of: pulse width, pulse amplitude, pulse frequency, and pulse burst duration.

13. The system of claim 1, further comprising a lead coupling the pulse generator to the nerve cuff.

14. The system of claim 1, further comprising an impedance sensing sub-system configured to determine the impedance measured from the one or more electrodes.

15. A system for applying a high-frequency nerve block, the system comprising:
an implantable nerve cuff comprising one or more electrodes, wherein the nerve cuff is configured to at least partially surround a region of a nerve;
a pulse generator configured to generate a high-frequency signal having a pulse profile;
a memory storing a normalized treatment dose value and an indicator of cross-sectional area of the region of the nerve that is at least partially surrounded by the nerve cuff;
a controller configured to determine the pulse profile based on the normalized treatment dose value, a cross-sectional area of the region of the nerve that is at least partially surrounded by the nerve cuff, and an impedance measured from the one or more electrodes, wherein the controller is configured to drive the pulse generator to deliver the high-frequency signal having the pulse profile from the one or more electrodes.

16. A method of applying a high-frequency nerve block to treat pain, the method comprising:
determining or receiving an indicator of a cross-sectional area of a region of a nerve at least partially surrounded by a nerve cuff having one or more electrodes;
determining an impedance value from one or more of the electrodes;
determining, in a controller, a pulse profile based on a normalized treatment dose value, the cross-sectional area of the region of the nerve at least partially surrounded by the nerve cuff, and the impedance value; and
applying a high-frequency signal having the pulse profile to the nerve.

17. The method of claim 16, further comprising applying the nerve cuff at least partially around the nerve.

18. The method of claim 16, wherein the high-frequency signal has a frequency of 1 kHz or greater.

19. The method of claim 16, wherein the high-frequency signal has a frequency of between 1 kHz and 100 kHz.

20. The method of claim 16, wherein the normalized treatment dose value is between 0.1 and 5 μC/phase/cm².

21. The method of claim 16, wherein the normalized treatment dose value is between 0.5 and 4 μC/phase/cm².

22. The method of claim 16, further comprising estimating the cross-sectional area of the region of a nerve at least partially surrounded by the nerve cuff from the indicator of the cross-sectional area of the region.

23. The method of claim 16, wherein determining the indicator of the cross-sectional area of the region of a nerve comprises measuring a circumference or a diameter of the region of the nerve that is or will be at least partially surrounded by the nerve cuff.

24. The method of claim 16, further comprising receiving a signal from a user to apply the high-frequency signal having the pulse profile to the nerve.

25. The method of claim 16, wherein determining the pulse profile comprises determining one or more of: pulse width, pulse amplitude, pulse frequency, and pulse burst duration.

26. The method of claim 16, wherein applying the high-frequency signal comprises applying the high-frequency signal for a treatment duration of greater than 10 minutes.

27. (canceled)
